(19) United States
(12) Patent Application Publication
(10) Pub. No.: US 2011/0159531 A1
(43) Pub. Date: Jun. 30, 2011

(54) METHOD AND APPARATUS FOR MEASURING AMOUNT OF SUBSTANCE

(75) Inventors: Sung Hwa LEE, Anyang-si (KR); Jong Rip LEE, Bucheon-si (KR); Hyug Rae CHO, Seoul (KR)

(73) Assignee: SAMSUNG ELECTRONICS CO., LTD., Suwon-si (KR)

(21) Appl. No.: 12/906,188

(22) Filed: Oct. 18, 2010

(30) Foreign Application Priority Data
Dec. 30, 2009 (KR) 10-2009-134271

(51) Int. Cl. C12Q 1/02; C12M 1/34

(52) U.S. Cl. 435/29; 435/288.7

(57) ABSTRACT

Disclosed herein are a method and apparatus for measuring an enzyme amount. The measurement of the enzyme amount is carried out by measuring a transmittance of a reaction mixture containing the enzyme and its substrate over the reaction time period and providing an optical characteristic curve, dividing the optical characteristic curve by a uniform distance in a reaction time axis direction to set a plurality of sections, and selecting one section satisfying preset linear conditions and having a maximum gradient absolute value from the plurality of sections and calculating an enzyme amount from the gradient of the optical characteristic curve in the selected section.

### CROSS-REFERENCE TO RELATED APPLICATION(S)

This application claims the benefit of Korean Patent Application No. 2009-0134271, filed on Dec. 30, 2009 in the Korean Intellectual Property Office, the disclosure of which is incorporated herein by reference.

### BACKGROUND

1. Field

Embodiments of the present invention relate to a method and apparatus for measuring an amount of an enzyme.

2. Description of the Related Art

Colorimetric analysis is a method for measuring an amount of an enzyme in a sample. This method is carried out by reacting an enzyme with a substrate, directly or indirectly obtaining an optical signal such as fluorescent light from a reaction product of the enzyme and the substrate and deciding an amount of enzyme in a sample through correlation between the optical signal strength and the enzyme amount.

For example, in accordance with a method for measuring an amount of a substance of interest in a sample using optical absorbance, a set of “known” samples, i.e., samples that contain the substance of interest in amounts which span the range of concentrations that are expected to be present in samples to be tested is prepared and assayed to produce a standard curve. Then, assay the test or unknown samples, followed by using the standard curve to find the corresponding substance concentration for each measurement obtained.

### SUMMARY

However, this method is disadvantageous in that a measurement of enzyme reaction degree may not be accurate or precise, or it is difficult to find a reaction section, when sudden variation (i.e., noise) occurs upon measurement of an absorbance curve as a function of time, the concentration of an enzyme in a sample is high, or a substrate is exhausted prior to a final measurement of the reaction. In particular, when a curve showing concentration variations (i.e., variations in transmittance or absorbance) of reaction product as a function of time includes a nonlinear section or a plurality of linear sections in a time section at which the enzyme reaction degree is measured, the measured enzyme reaction degree is unreliable.

Therefore, it is an aspect of the present invention to accurately identify the most linear section of a curve showing concentration variations (that is, variations in transmittance or absorbance) of reaction product as a function of time in a time section, at which the enzyme reaction degree is measured, and thereby accurately measure the enzyme reaction degree from the most linear section.

Additional aspects of the invention will be set forth in part in the description which follows and, in part, will be obvious from the description, or may be learned by practice of the invention.

In accordance with one aspect of the present invention, a method for measuring an enzyme amount, includes: irradiating light to a reaction mixture containing an enzyme and a substrate thereof over a certain period of time to obtain an optical characteristic curve showing a variation in reaction degree as a function of reaction time; dividing the optical characteristic curve along the reaction time axis to set a plurality of first sections of a uniform distance; selecting one first section satisfying preset linear conditions and having a maximum gradient absolute value from the plurality of first sections; and calculating an enzyme amount from the gradient of the optical characteristic curve in the selected first section based on a preset values.

The setting of the plurality of first sections may include: dividing the optical characteristic curve along the time axis to set a plurality of second sections of an uniform distance; and combining two or more adjacent second sections to set an individual first section of the plural first sections.

The first sections may be set such that the distance between the adjacent first sections is the same as the length of one second section and the first sections are successive.

In an embodiment, the method for measuring an amount of a substance of interest in a sample, includes: irradiating light to a container which contains the substance of interest and a second substance that is capable of reacting with the substrate of interest, wherein a reaction of the substance of interest and the second substance occurs in the container; plotting an optical characteristic curve in two crossing axes, wherein one of the two axes shows a measured transmittance or absorbance and the other axis shows a reaction time passed; dividing the optical characteristic curve along the reaction time axis to set a plurality of first sections of a first uniform distance; and selecting one first section satisfying preset linear conditions and having a maximum gradient absolute value and calculating the amount of the substance of interest from the gradient of the optical characteristic curve in the selected first section.

In the above-described method, the setting of the plurality of first sections may include: dividing the optical characteristic curve along the time axis to set a plurality of second sections of a second uniform distance, wherein the second uniform distance is smaller than the first uniform distance; combining two or more adjacent second sections to set one first section; and combining two or more adjacent second sections to set another first section in a way that each of the first sections spans different regions of the optical characteristic curve.

At least one of the adjacent second sections combined to set the another first section is also included in the one first section, so that the one first section and the another first section span partially overlapping regions of the optical characteristic curve, and wherein the distance between the start point of the one first section along the reaction time axis and the start point of the another first section along the reaction time axis is identical to the second uniform distance.

The calculation of the enzyme amount may include: selecting one first section having a maximum gradient absolute value from the plurality of first sections; and calculating an enzyme amount from the gradient of the optical characteristic curve in the selected first section, when the selected first section is linear. The enzyme amount may be a variable of an amount of its substrate or an amount of a product produced by the reaction between the enzyme and the substrate, wherein the amount of the substrate and/or the amount of the reaction product change over the period of reaction time, and wherein the measured transmittance or absorbance of the container through which the light passes varies depending on the amount of the substrate or the amount of the reaction product.

The calculation of the enzyme amount may include: selecting one first section having a maximum gradient absolute value from the plurality of first sections; selecting a second section having a maximum gradient absolute value from the selected first section, when the optical characteristic curve of the selected first section is nonlinear; selecting all adjacent second sections having a gradient satisfying preset linear conditions with respect to the gradient of the selected second section; and calculating an enzyme amount from an entire gradient of all the second sections satisfying preset linear conditions.

The decision of linear conditions of the second sections in the selected first section may include: selecting a second section having a maximum gradient absolute value from the selected first section; comparing a gradient of the selected second section with each gradient of other second sections adjacent thereto; and deciding the optical characteristic curve to be non-linear, when the value of a gradient having a higher absolute value divided by a gradient having a low absolute value, among gradients of the second sections, is lower than a set reference value, and deciding the optical characteristic curve to be linear, when the value of a gradient having a higher absolute value divided by a gradient having a low absolute value is higher than the set reference value.

The optical characteristic curve may be obtained based on variation in transmittance or absorbance according to reaction of the enzyme in the sample cell containing the enzyme and the substrate.

In accordance with another aspect of the present invention, an apparatus for measuring an enzyme amount includes: a sample cell containing an enzyme and a substrate; a light source to irradiate light to the sample cell; a light detector to generate a light detection signal corresponding to transmittance or absorbance in the sample cell; and an enzyme amount calculator to generate an optical characteristic curve which is a reaction product absorbance or transmittance along one of two intersecting axis and a reaction time along the other axis, said optical characteristic curve indicating a variation in reaction degree as a function of time and to divide the optical characteristic curve along the reaction time axis to set a plurality of first sections of a uniform distance, and to select one first section satisfying preset linear conditions and having a maximum gradient absolute value from the plurality of first sections and calculate an enzyme amount from the gradient of the optical characteristic curve in the selected first section.

The enzyme amount calculator may set the first sections by dividing the optical characteristic curve by a preset distance in a time axis direction to set a plurality of second sections, and combining two or more adjacent second sections to set an individual section of the plural first sections.

The enzyme amount calculator may set the first sections such that the distance between the adjacent first sections is the same as the length of one second section and the first sections are successive.

The enzyme amount calculator may calculate the enzyme amount by selecting one first section having a maximum gradient absolute value from the plurality of first sections, and calculating an enzyme amount from the gradient of the optical characteristic curve in the selected first section, when the optical characteristic curve of the selected first section is linear.

The enzyme amount calculator may calculate the enzyme amount by selecting one first section having a maximum gradient absolute value from the plurality of first sections, selecting a second section having a maximum gradient absolute value from the selected first section, when the optical characteristic curve of the selected first section is nonlinear, selecting all adjacent second sections satisfying preset linear conditions with respect to the gradient of the selected second section, and calculating an enzyme amount from an entire gradient of all the second sections, in the selected first section, satisfying preset linear conditions.

The enzyme amount calculator may decide linear conditions of the second sections in the selected first section by selecting a second section having a maximum gradient absolute value from the selected first section; comparing a gradient of the selected second section with each gradient of other second sections adjacent thereto; and deciding the optical characteristic curve to be non-linear, when the value of a gradient having a higher absolute value divided by a gradient having a low absolute value, among gradients of second sections, is lower than a set reference value, and deciding the optical characteristic curve to be linear, when the value of a gradient having a higher absolute value divided by a gradient having a low absolute value is higher than the set reference value.

The enzyme amount calculator may obtain the optical characteristic curve based on the variation in transmittance or absorbance according to reaction of the enzyme in the sample cell containing the enzyme and the substrate thereof

According to another embodiment, an apparatus for measuring an amount of a substance of interest is disclosed, which apparatus includes: a sample cell containing the substance of interest and a second substance that is capable of reacting with the substance of interest; a light source to irradiate light to the sample cell; a light detector to generate a light detection signal corresponding to transmittance or absorbance of the sample cell; and a substance amount calculator to generate an optical characteristic curve in two crossing axes, in which one of the two crossing axes indicates a transmittance or absorbance of the sample cell and the other axes indicates a reaction time, to divide the optical characteristic curve along the reaction time axis to set a plurality of first sections of a first uniform distance, and to select one first section satisfying preset linear conditions and having a maximum gradient absolute value, and to calculate the amount of the substance of interest from the gradient of the optical characteristic curve in the selected first section.

The setting of the first sections includes: dividing the optical characteristic curve along the time axis to set a plurality of second sections of a second uniform distance; wherein the second uniform distance is smaller than the first uniform distance; combining two or more adjacent second sections to set one first section; and combining two or more adjacent second sections to set another first section in a way that each of the first sections spans different regions of the optical characteristic curve. At least one of the adjacent second sections combined to set the another first section is also included in the one first section, so that the one first section and the another first section span partially overlapping regions of the optical characteristic curve, and wherein the distance between the start point of the one first section along the reaction time axis and the start point of the another first section along the reaction time axis is identical to the second uniform distance.

### BRIEF DESCRIPTION OF THE DRAWINGS

These and/or other aspects of embodiments will become apparent and more readily appreciated from the following description of the embodiments, taken in conjunction with the accompanying drawings of which:

FIG. 1 is an apparatus for measuring an amount of an enzyme according to one embodiment of the present invention;

FIG. 2 illustrate various optical characteristic curves (in particular, optical characteristic curves showing an absorbance variation as a function of reaction time) obtained by measuring absorbance of a reaction product of an enzyme and a substrate; and

FIGS. 3 and 5 illustrate a method for measuring an enzyme amount according to one embodiment of the present invention, and

FIG. 4 is a schematic diagram illustrating the blocks 310 and 312 shown in FIG. 3 in more detail.

### DETAILED DESCRIPTION

Reference will now be made in detail to the embodiments, examples of which are illustrated in the accompanying drawings, wherein like reference numerals refer to like elements throughout. The description is made with respect to an enzyme quantification; however, an amount of any substance of interest may also be determined by the methods and apparatus according to embodiments.

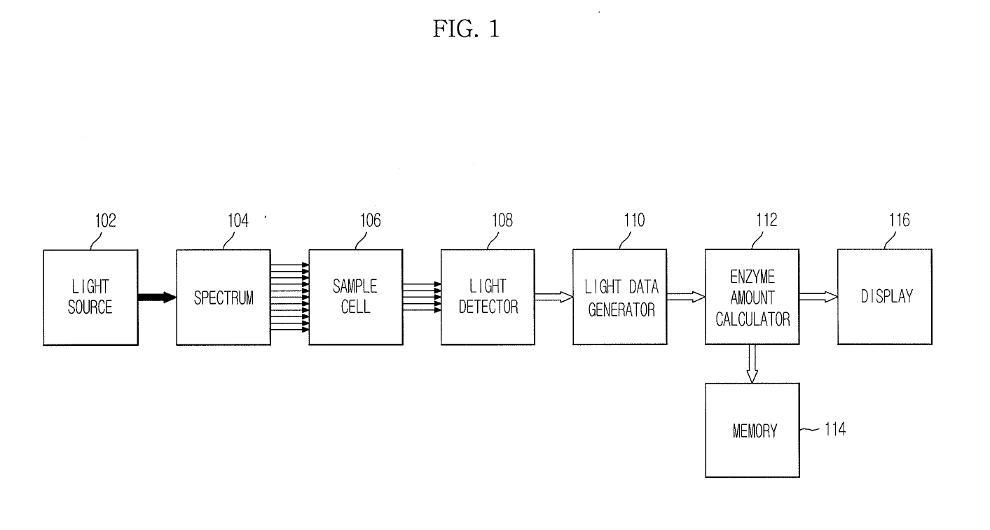

FIG. 1 is an apparatus for measuring an amount of an enzyme according to one embodiment of the present invention. As shown in FIG. 1, the apparatus comprises a light source 102, a spectrum 104, a sample cell 106, a light detector 108, a light data generator 110 and an enzyme amount calculator 112. Light with a predetermined wavelength (for example, 340 nm) from the light source 102 is broadly diffused from the spectrum 104 and uniformly distributed to the sample cell 106. The sample cell 106 contains an enzyme and a substrate thereof so that there an enzyme-substrate reaction occurs. The substance to be quantified according to the embodiments is not limited to an enzyme, but may include any substance of interest. In an embodiment, the substance of interest is coexist in a sample cell 106 with a second substance which is capable of specifically or non-specifically reacting with the substance of interest. As the reaction proceeds, the concentration of reaction product is varied and transmittance (or absorbance) of light is also varied according to the concentration variation. The light detector 108 detects an amount of light transmitted in the sample cell 106, generates a light detection signal corresponding to the detected light-transmitted amount and supplies the signal to the light data generator 110. The light detection signal generated in the light detector 108 is fundamentally based on the transmittance of the sample cell 106, but an absorbance may be obtained from a ratio between an amount of light irradiated from the light source 102 and an amount of light transmitted to the sample cell 106. The light data generator 110 generates light data indicating light transmittance or absorbance from the intensity of the light detection signal supplied from the light detector 108 and supplies the light data to the enzyme amount calculator 112. The enzyme amount calculator 112 continuously or periodically (e.g., at an interval of 0.2 seconds) receives the light data from the light data generator 110 to plot an optical characteristic curve, indicating a variation in transmittance or absorbance according to variation in the concentration of the reaction product of the enzyme and the substrate in the sample cell 106 for a predetermined period (for example, 50 seconds). The enzyme amount calculator 112 calculates and outputs an amount of an enzyme present in the sample cell 106 from a gradient that satisfies preset linear conditions and has a maximum absolute value in the optical characteristic curve. The optical characteristic curve and the enzyme amount obtained by calculation of the enzyme amount calculator 112 are stored in a memory 114 and are displayed on a display 116, to allow a user to visually confirm concentration variations of the reaction product in the sample cell 106. The display 116 shows not only the optical characteristic curve formed from the enzyme amount calculator 112, but also a portion corresponding to a gradient that satisfies preset linear conditions and has a maximum absolute value in the optical characteristic curve.

The variation in transmittance or absorbance of the sample cell 106 may be based on changes in the amount of the substrate consumed through the reaction or the amount of the reaction product produced by the reaction in the sample cell. The wavelength of the light supplied from the light source 102 may be determined based on the substance of which amount changes over the predetermined period of time, for example due to a reaction thereof with a second substance coexisting in the sample cell. An appropriate wavelength may be readily determined by one skilled in the art. In the present disclosure, as a non-limiting example, an enzyme and a substrate thereof are exemplified and the transmittance (or absorbance) of a product of a reaction of the enzyme and the substrate is explained.

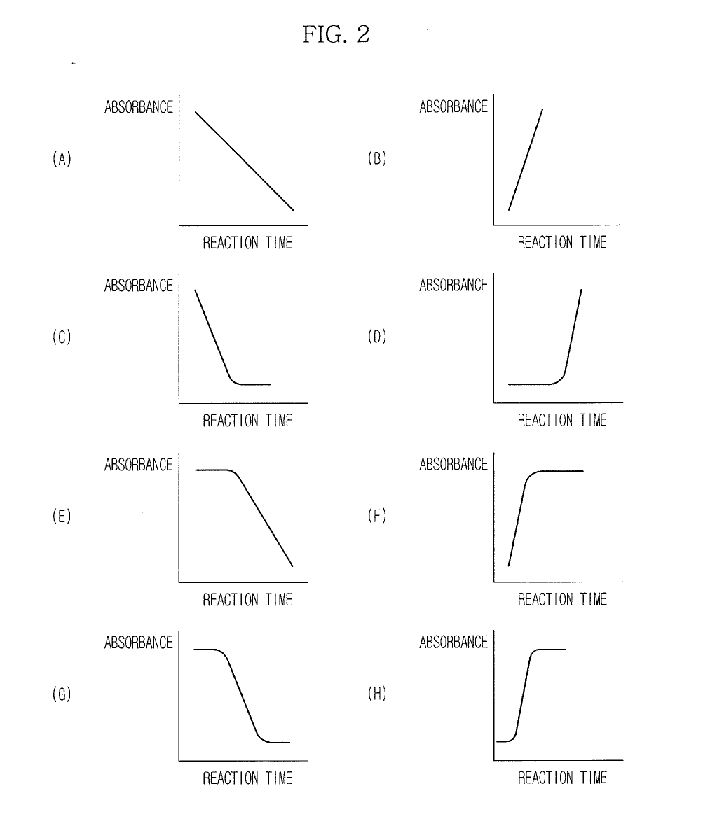

FIG. 2 illustrates various forms of optical characteristic curves (in particular, optical characteristic curves showing absorbance variation as a function of reaction time) obtained by measuring a change in the absorbance of a reaction product of an enzyme and a substrate. In FIG. 2, (A) and (B) include a single linear section only, (C) (D), (E) and (F) include two linear sections, and (G) and (H) include three linear sections. As shown in (C) to (H) of FIG. 2, a gradient of the optical characteristic curve according to the reaction of enzyme/substrate is irregular or involves a plurality of linear sections. This irregular or heterogeneous reaction pattern may be caused when the enzyme and the substrate are not uniformly mixed or are not present in a suitable ratio.

The gradient of linear sections of the optical characteristic curve obtained from the transmittance or absorbance of the reaction product of the enzyme and the substrate is a basis for calculating the amount of the enzyme. That is, an actual amount of enzyme can be determined by comparing the optical characteristic curve (in particular, a linear section) obtained from the measured absorbance or transmittance with a reference (or standard) curve (in particular, a linear section) obtained from known amounts of the enzyme and the substrate. For this reason, to more accurately measure the enzyme amount, a section which is the most linear and has a maximum gradient absolute value in the optical characteristic curve should be found.

Herein, in the case of optical characteristic curves consisting of only one linear section, as shown in (A) and (B) of FIG. 2, an enzyme amount is calculated from a standard curve (or a predetermined overall gradient of the optical characteristic curve), while in the case of optical characteristic curves including a plurality of linear sections, as shown in (C) to (H) of FIG. 2, an enzyme amount is calculated from the linear section which is the most linear and has a maximum gradient absolute.

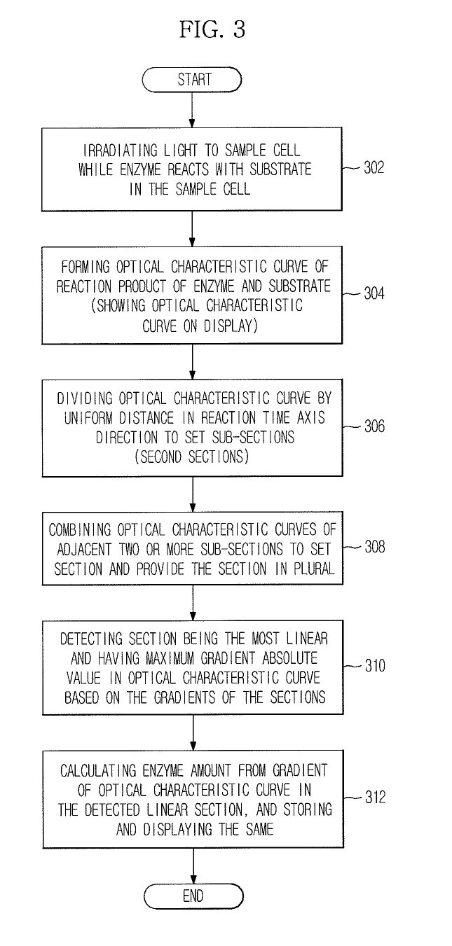

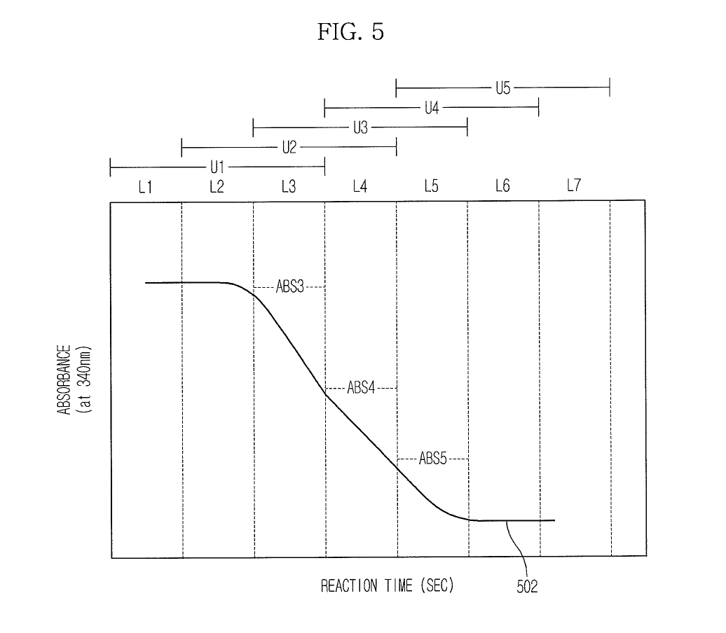

FIGS. 3 to 5 illustrate a method for measuring an enzyme amount according to one embodiment of the present invention. As shown in FIG. 3, in accordance with the method, first, light is irradiated to a sample cell 106 where a reaction between a substance of interest (e.g., enzyme) to be tested for its amount, and a second substance (e.g., substrate of the enzyme) which is capable of reacting with the substance of interest occurs (302). As mentioned in the description of the enzyme amount calculator 112 with reference to FIG. 1, the enzyme amount calculator 112 generates an optical characteristic curve, indicating a concentration variation in reaction product based on light data of the reaction product of an enzyme and a substrate (304), by employing the measured values of transmittance or absorbance of the light through the sample cell 106. The wavelength of the light may be determined based on the reaction product or the second substrate of which amount decreases as it is consumed as the reaction progresses. Assuming that the optical characteristic curve is an optical characteristic curve 502 shown in FIG. 5, the optical characteristic curve 502 is divided along the reaction time axis to set a plurality of sub-sections (second sections, L1, L2, L3, . . . ) of an uniform distance (i.e., L1=L2=L3 . . . L6=L7) (306). Referring to FIG. 3, two or more adjacent sub-sections, L1, L2, . . . L7 of the optical characteristic curve are combined to set a plurality of sections, U1, U2, . . . U5 (308). As shown in FIG. 5, the sections may be set by grouping two or more (e.g., three in FIG. 5) adjacent sub-sections such that the distance between the start point of a section and the start point of its successive section is the same as one sub-section. That is, section U1 is formed by combining sub-sections L1, L2 and L3, section U2 is formed by combining sub-sections L2, L3, and L4, section U3 is formed by combining sub-sections L3, L4, and L5, etc., and the distance between the start point of section U1 and the start pint of section U2 is identical to L1.

Referring to FIG. 3, the enzyme amount calculator 112 identifies the section which is the most linear and has a maximum gradient absolute value in the optical characteristic curve 502 based on the gradients of all of the sections (310). Also, the enzyme amount calculator 112 determines an enzyme amount from the gradient of the identified most linear section, by comparing it with a predetermined standard gradient, stores the amount in a memory 114, and shows the amount on a display 116 (312).

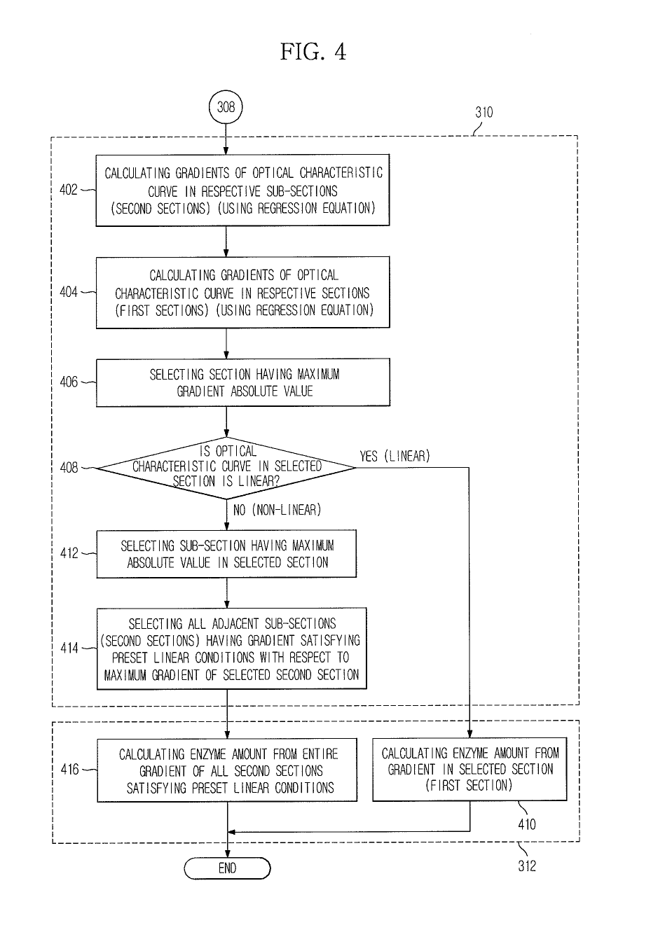

FIG. 4 is a schematic diagram illustrating the blocks 310 and 312 shown in FIG. 3 in more detail. As shown in FIG. 4, absolute values of gradients of the optical characteristic curve 502 in respective sub-sections (L1, L2, L3, . . . L7 in FIG. 5) are calculated (using regression equation) (402). Also, absolute values of gradients of the optical characteristic curve 502 in respective sections (sections U1, U2, . . . U5 in FIG. 5) are calculated (using regression equation) (404). After gradients of the optical characteristic curve 502 in the sub-sections (L1 . . . L7) and sections (U1 . . . U5) are obtained, a section having a maximum gradient absolute value is selected from the plurality of sections (406).

Assuming section U3 is identified as the section having a maximum gradient absolute value in the optical characteristic curve 502 shown in FIG. 5, the enzyme amount calculator 112 decides linearity of the selected section U3 to calculate an enzyme amount from the gradient of the selected section U3 (408). When the optical characteristic curve 502 in the selected section U3 is linear (“Yes” of 408), the enzyme amount calculator 112 calculates an enzyme amount from the gradient of the selected section U3 (410) by, for example, comparing it with a standard (or reference) gradient chart or a standard (or reference) curve of known enzyme amounts (not shown). On the other hand, the selected section U3 is non-linear (“No” of 408), the enzyme amount calculator 112 performs an additional process for searching a section satisfying preset linear conditions in the optical characteristic curve 502 of the selected section U3.

That is, when the optical characteristic curve 502 of the selected section U3 is non-linear (“No” of 408), the enzyme amount calculator 112 searches and selects a sub-section having a maximum absolute value from the sub-sections L3 to L5 included in the selected section U3 (412). For example, in the optical characteristic curve 502 of FIG. 5, the first sub-section L3 of the section U3 has the highest gradient. The enzyme amount calculator 112 compares the gradient of the sub-section L3 with those of adjacent successive sub-sections L4 and L5 and determines whether or not the sub-section L3 and the remaining two sub-sections L4 and L5 satisfy the preset linear conditions (414). For this purpose, the enzyme amount calculator 112 determines whether an absolute vale ratio of the sub-section L3 and another sub-section L4 adjacent thereto satisfies a preset reference value. For example, assuming that absolute values of the gradients of three sub-sections L3, L4 and L5 are ABS3, ABS4 and ABS5, respectively, and a set reference value is 0.8, if ABS4<ABS3 and ABS4/ABS3>0.8, two sub-sections L3 and L4 are considered to satisfy preset linear conditions. On the other hand, if the above conditions are not met, L3 and L4 are considered to not satisfy the preset linear conditions. When the two sub-sections L3 and L4 are considered to not satisfy preset linear conditions, only the sub-section L3 is considered to be a linear section and an enzyme amount is calculated based on the gradient of the sub-section L3 alone. On the other hand, when the two sub-sections L3 and L4 are considered to satisfy preset linear conditions, a process to decide linearity of the sub-sections with another sub-section L5 is further performed. That is, when ABS5<ABS3 and ABS5/ABS3>0.8, two sub-sections L3 and L5 are considered to satisfy preset linear conditions. If the above conditions are not met, the sub-sections L3 and L5 are considered to not satisfy the preset linear conditions. When two sub-sections L3 and L5 are considered to not satisfy preset linear conditions, an enzyme amount is calculated based on the entire gradient of two sub-sections L3 and L4, which are considered to satisfy the preset linear conditions. On the other hand, when all of three sub-sections L3 to L5 are considered to satisfy the preset linear conditions, an enzyme amount is calculated based on the entire gradient of the three sub-sections.

In accordance with one embodiment of the present invention, although an optical characteristic curve showing concentration variations (i.e., variations in transmittance or absorbance) of a reaction product as a function of time includes a nonlinear section or a plurality of linear sections, the most linear section can be accurately searched, enzyme reaction degree can be accurately obtained from the most linear section, and an amount or reaction rate of enzyme can thus be accurately obtained therefrom.

Although a few embodiments of the present invention have been shown and described, it would be appreciated by those skilled in the art that changes may be made in these embodiments without departing from the principles and spirit of the invention, the scope of which is defined in the claims and their equivalents.

What is claimed is:

1. A method for measuring an amount of a substance of interest in a sample, comprising:
irradiating light to a container which contains the substance of interest and a second substance that is capable of reacting with the substrate of interest, wherein a reaction of the substance of interest and the second substance occurs in the container;
plotting an optical characteristic curve in two crossing axes, wherein one of the two axes shows a measured transmittance or absorbance and the other axis shows a reaction time passed;
dividing the optical characteristic curve along the reaction time axis to set a plurality of first sections of a first uniform distance; and
selecting one first section satisfying preset linear conditions and having a maximum gradient absolute value and calculating the amount of the substance of interest from the gradient of the optical characteristic curve in the selected first section.

2. The method according to claim 1, wherein the setting of the plurality of first sections comprises:
dividing the optical characteristic curve along the time axis to set a plurality of second sections of a second uniform distance, wherein the second uniform distance is smaller than the first uniform distance;
combining two or more adjacent second sections to set one first section; and
combining two or more adjacent second sections to set another first section in a way that each of the first sections spans different regions of the optical characteristic curve.

3. The method according to claim 2, wherein at least one of the adjacent second sections combined to set the another first section is also included in the one first section, so that the one first section and the another first section span partially overlapping regions of the optical characteristic curve, and wherein the distance between the start point of the one first section along the reaction time axis and the start point of the another first section along the reaction time axis is identical to the second uniform distance.

4. The method according to claim 1, wherein the calculation of the amount of the substance of interest comprises:
selecting one first section having a maximum gradient absolute value; and
calculating the amount from the gradient of the optical characteristic curve in the selected first section, when the selected first section is linear, by comparing the gradient with a predetermined reference gradient.

5. The method according to claim 1, wherein the calculation of the enzyme amount comprises:
selecting one first section having a maximum gradient absolute value;
selecting a second section having a maximum gradient absolute value from the selected first section, when the optical characteristic curve of the selected first section is nonlinear;
selecting all adjacent second sections having a gradient satisfying preset linear conditions with respect to the gradient of the selected second section; and
calculating the amount of the substance of interest from the entire gradient of all of the second sections satisfying the preset linear conditions.

6. The method according to claim 5, wherein the decision of linear conditions of the second sections in the selected first section comprises:
selecting a second section having a maximum gradient absolute value from the selected first section;
comparing the gradient of the selected second section with each gradient of other second sections adjacent thereto; and
deciding whether the other second sections be non-linear, when the value of a gradient having a higher absolute value divided by a gradient having a low absolute value, among gradients of the second sections, is lower than a preset reference value, and deciding the optical characteristic curve to be linear, when the value of a gradient having a higher absolute value divided by a gradient having a low absolute value is higher than the preset reference value.

7. The method according to claim 1, wherein the amount of the second substance or the amount of a product produced by reaction between the substance of interest and the second substance in the container changes over the period of reaction time, and wherein the measured transmittance or absorbance of the container through which the light passes varies depending on the amount of the second substance or the amount of the reaction product.

8. An apparatus for measuring an amount of a substance of interest, comprising:
a sample cell containing the substance of interest and a second substance that is capable of reacting with the substance of interest;
a light source to irradiate light to the sample cell;
a light detector to generate a light detection signal corresponding to transmittance or absorbance of the sample cell; and
a substance amount calculator to generate an optical characteristic curve in two crossing axes, in which one of the two crossing axes indicates a transmittance or absorbance of the sample cell and the other axes indicates a reaction time, to divide the optical characteristic curve along the reaction time axis to set a plurality of first sections of a first uniform distance, and to select one first section satisfying preset linear conditions and having a maximum gradient absolute value, and to calculate the amount of the substance of interest from the gradient of the optical characteristic curve in the selected first section.

9. The apparatus according to claim 8, wherein the setting of the first sections comprises:
dividing the optical characteristic curve along the time axis to set a plurality of second sections of a second uniform distance,
wherein the second uniform distance is smaller than the first uniform distance;
combining two or more adjacent second sections to set one first section; and
combining two or more adjacent second sections to set another first section in a way that each of the first sections spans different regions of the optical characteristic curve.

10. The apparatus according to claim 9, wherein at least one of the adjacent second sections combined to set the another first section is also included in the one first section, so that the one first section and the another first section span partially overlapping regions of the optical characteristic curve, and wherein the distance between the start point of the one first section along the reaction time axis and the start point of the another first section along the reaction time axis is identical to the second uniform distance.

11. The apparatus according to claim 8, wherein the calculation of the amount of substance of interest comprises:
selecting one first section having a maximum gradient absolute value; and
calculating the amount from the gradient of the optical characteristic curve in the selected first section, when the optical characteristic curve of the selected first section is linear.

12. The apparatus according to claim 8, wherein the calculation of the enzyme amount comprises:
selecting one first section having a maximum gradient absolute value;
selecting one second section in the selected first section, said one second section having a maximum gradient absolute value from the selected first section, when the optical characteristic curve of the selected first section is nonlinear;
selecting all adjacent second sections having a gradient satisfying preset linear conditions with respect to the gradient of the selected second section; and
calculating the amount of the substance of interest from an entire gradient of all the second sections satisfying preset linear conditions.

13. The apparatus according to claim 12, wherein the decision of linear conditions of the second sections in the selected first section comprises:
selecting a second section having a maximum gradient absolute value;
comparing the gradient of the selected second section with each gradient of other second sections adjacent thereto; and
deciding whether the other second sections be non-linear, when the value of a gradient having a higher absolute value divided by a gradient having a low absolute value, among gradients of second sections, is lower than a preset reference value, and deciding the optical characteristic curve to be linear, when the value of a gradient having a higher absolute value divided by a gradient having a low absolute value is higher than the preset reference value.

14. The apparatus according to claim 8, wherein the amount of the second substance or the amount of a product produced by a reaction of the substance of interest and the second substance, in the sample cell changes over the period of reaction time, and wherein the measured transmittance or absorbance of the container through which the light passes varies depending on the amount of the second substance or the amount of the reaction product.
